(19) United States
(12) Patent Application Publication
(10) Pub. No.: US 2014/0004071 A1
(43) Pub. Date: Jan. 2, 2014

(54) Thermochromic Dental Material

(71) Applicant: William Steven Brown, Aledo, TX (US)

(72) Inventor: William Steven Brown, Aledo, TX (US)

(21) Appl. No.: 13/931,141

(22) Filed: Jun. 28, 2013

Related U.S. Application Data
(60) Provisional application No. 61/666,823, filed on Jun. 30, 2012.

(51) Int. Cl. A61K 6/00

(52) U.S. Cl. CPC A61K 6/0064
USPC 424/78.02; 523/115

(57) ABSTRACT

The present invention includes a thermochromic dental material comprising a polymeric dental composition; and a temperature sensitive thermochromic dye in contact with the polymeric dental composition, wherein the temperature sensitive thermochromic dye is colorless at body temperature and undergoes a reversible color change at a temperature of between 99-160° F.

### CROSS-REFERENCE TO RELATED APPLICATIONS

This Non-Provisional Patent Application claims priority to U.S. Provisional Patent Application Ser. No. 61/666,823, filed Jun. 30, 2012, the contents of which is incorporated by reference herein in its entirety.

### TECHNICAL FIELD OF THE INVENTION

The present invention relates generally to methods and compositions for dental materials and in particular, to dental materials containing a thermochromic dye which undergoes a reversible color change as a function of temperature changes at higher temperatures.

### STATEMENT OF FEDERALLY FUNDED RESEARCH

None.

### INCORPORATION-BY-REFERENCE OF MATERIALS FILED ON COMPACT DISC

None.

### BACKGROUND OF THE INVENTION

Without limiting the scope of the invention, its background is described in connection with dental materials containing a thermochromic dye.

The dental procedures relating to dental implant treatments including implant placement and particularly to restoration of implants there are a number of critical factors that can affect the efficacy of implant treatment. Implant success depends on whether the implant and bone can handle the forces, stresses, and biological demands created by the implant, the environment, and both the physical and biological loads. For example, one factor that determines the success of the implant is the amount of heat generated and transmitted to the bone and surrounding tissue during the implant treatment. Excess heating of the implant can result in heat transfer to the bone and surrounding tissue and cell damage and/or cell death can result. However, currently there is no mechanism to determine the amount of heat generated and transmitted to the implant, bone and surrounding tissue.

U.S. Pat. No. 6,670,436 (the '436 Patent) discloses a thermochromic dental material which contain a thermochromic dye that display a reversible change of color in the event of changes in temperature, so that they can be distinguished from the natural dentin.

### SUMMARY OF THE INVENTION

In one embodiment the present invention provides a thermochromic dental material comprising a polymeric dental composition; and a temperature sensitive thermochromic dye in contact with the polymeric dental composition, wherein the temperature sensitive thermochromic dye is colorless at body temperature and undergoes a reversible color change at a temperature of between 99-160° F. The temperature sensitive thermochromic dye is a visual indicator a temperature of about 99, 100, 101, 102, 103, 105, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160° F., or above. The temperature sensitive thermochromic dye may be selected from bisphenol A, tetrabromobisphenol A, gallnut acid, salicylic acid, 3-isopropyl salicylate, 3-cyclohexyl salicylate, 3-5-di-tert-butyl salicylate, 3,5-di-α-methyl benzyl salicylate, 4,4′-isopropylidenediphenol, 1,1′-isopropylidene bis (2-chlorophenol), 4,4′-isopropylene bis (2,6-dibromophenol), 4,4′-isopropylidene bis (2,6-dichlorophenol), 4,4′-isopropylidene bis(2-ethyl phenol), 4,4′-isopropylidene bis(2,6-dimethyl phenol), 4,4′-isopropylidene bis (2-tert-butyl phenol), 4,4′-sec-butylidene diphenol, 4,4′-cyclohexylidene bisphenol, 4,4′-cyclohexylidene bis (2-ethyl phenol), 4-tert-butyl phenol, 4-phenyl phenol, 4-hydroxy diphenoxide, α-naphthol, β-naphthol, 3,5-xylenol, thymol, methyl-4-hydroxybenzoate, 4-hydroxyacetophenone, novolak phenol resins, 2,2′-thio bis(4,6-dichloro phenol), catechol, resorcin, hydroxynone, hydroquinone, pyrogallol, fluoroglycine, fluoroglycine carbonate, 4-tert-octyl catechol, 2,2′-methylene bis (4-chlorophenol), 2,2′-methylene bis (4-methyl-6-tert-butyl phenol), 2,2′-dihydroxy diphenyl, ethyl p-hydroxybenzoate, propyl p-hydroxybenzoate, butyl p-hydroxybenzoate, benzyl p-hydroxybenzoate, p-hydroxybenzoate-p-chlorobenzyl, p-hydroxybenzoate-o-chlorobenzyl, p-hydroxybenzoate-p-methylbenzyl, p-hydroxybenzoate-n-octyl, benzoic acid, zinc salicylate, 1-hydroxy-2-naphthoic acid, 2-hydroxy-6-naphthoic acid, 2-hydroxy-6-zinc naphthoate, 4-hydroxy diphenyl sulphone, 4-hydroxy-4′-chloro diphenyl sulfone, bis (4-hydroxy phenyl) sulfide, 2-hydroxy-p-toluic acid, 3,5-di-tert-zinc butyl salicylate, 3,5-di-tert-tin butyl salicylate, tartaric acid, oxalic acid, maleic acid, citric acid, succinic acid, stearic acid, 4-hydroxyphthalic acid, boric acid, thiourea derivative, 4-hydroxy thiophenol derivative, bis(4-hydroxyphenyl) acetate, bis(4-hydroxyphenyl)ethyl acetate, bis (4-hydroxyphenyl acetate-n-propyl, bis (4-hydroxyphenyl) acetate-n-butyl, bis (4-hydroxyphenyl) phenyl acetate, bis (4-hydroxyphenyl) benzyl acetate, bis (4-hydroxyphenyl) phenethyl acetate, bis (3-methyl-4-hydroxyphenyl) acetate, bis(3-methyl-4-hydroxyphenyl) methyl acetate, bis (3-methyl-4-hydroxyphenyl) acetate-n-propyl, 1,7-bis(4-hydroxyphenylthio) 3,5-dioxaheptane, 1,5-bis(4-hydroxyphenylthio) 3-oxaheptane, 4-hydroxy phthalate dimethyl, 4-hydroxy-4′-methoxy phenyl sulfone, 4-hydroxy-4′-ethoxy diphenyl sulfone, 4-hydroxy-4′-isopropoxy diphenyl sulfone, 4-hydroxy-4′-propoxy diphenyl sulfone, 4-hydroxy-4′-butoxy diphenyl sulfone, 4-hydroxy-4′-isopropoxy diphenyl sulfone, 4-hydroxy-4′-sec-butoxy diphenyl sulfone, 4-hydroxy-4′-tert-butoxy diphenyl sulfone, 4-hydroxy-4′-benzyloxy diphenyl sulfone, 4-hydroxy-4′-phenoxy diphenyl sulfone, 4-hydroxy-4′-(m-methyl benzoxy) diphenyl sulfone, 4-hydroxy-4′-(p-methyl benzoxy) diphenyl sulfone, 4-hydroxy-4′-(o-methyl benzoxy) diphenyl sulfone, 4-hydroxy-4′-(p-chloro benzoxy) diphenyl sulfone, 4-hydroxy-4′-oxyaryl diphenyl sulfone and the like. The temperature sensitive thermochromic dye may be bisphenol A. The temperature sensitive thermochromic dye may be from 0.01 to 10 wt %. The polymeric dental composition may be a dental composite, a dental acrylic, a polymer, or a plastic.

The polymeric dental composition comprises: between 20 to 90 wt % of one or more polymerizable monomers; 0.01 to 20 wt % of a polymerization initiator; and 5 to 90 wt % of a filler. The polymeric dental composition may be formed into a coating, a film, a filling, a reduction coping, a reduction coping, abutment, or a prosthetic.

In one embodiment the present invention provides a thermochromic coating comprising: a polymeric dental composition; and a temperature sensitive thermochromic dye in contact with the polymeric dental composition, wherein the temperature sensitive thermochromic dye is colorless at body temperature and undergoes a reversible color change at a temperature of between 99-160° F.

In one embodiment the present invention provides method of making a temperature indicating dental material by providing one or more polymerizable monomers for use in a dental composition; mixing one or more temperature sensitive thermochromic dye with the one or more polymerizable monomers; forming a dental composition; and curing the dental composition, wherein the temperature sensitive thermochromic dye is colorless at body temperature and undergoes a reversible color change at a temperature of between 99-160° F. The dental composition may be a reduction coping, a reduction coping, abutment, or a prosthetic. The temperature sensitive thermochromic dye may be a visual indicator a temperature of about 99, 100, 101, 102, 103, 105, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160° F., or above. The temperature sensitive thermochromic dye may be selected from bisphenol A, tetrabromobisphenol A, gallnut acid, salicylic acid, 3-isopropyl salicylate, 3-cyclohexyl salicylate, 3-5-di-tert-butyl salicylate, 3,5-di-α-methyl benzyl salicylate, 4,4′-isopropylidenediphenol, 1,1′-isopropylidene bis (2-chlorophenol), 4,4′-isopropylene bis (2,6-dibromophenol), 4,4′-isopropylidene bis (2,6-dichlorophenol), 4,4′-isopropylidene bis(2-ethyl phenol), 4,4′-isopropylidene bis(2,6-dimethyl phenol), 4,4′-isopropylidene bis (2-tert-butyl phenol), 4,4′-sec-butylidene diphenol, 4,4′-cyclohexylidene bisphenol, 4,4′-cyclohexylidene bis (2-ethyl phenol), 4-tert-butyl phenol, 4-phenyl phenol, 4-hydroxy diphenoxide, α-naphthol, β-naphthol, 3,5-xylenol, thymol, methyl-4-hydroxybenzoate, 4-hydroxyacetophenone, novolak phenol resins, 2,2′-thio bis(4,6-dichloro phenol), catechol, resorcin, hydroxynone, hydroquinone, pyrogallol, fluoroglycine, fluoroglycine carbonate, 4-tert-octyl catechol, 2,2′-methylene bis (4-chlorophenol), 2,2′-methylene bis (4-methyl-6-tert-butyl phenol), 2,2′-dihydroxy diphenyl, ethyl p-hydroxybenzoate, propyl p-hydroxybenzoate, butyl p-hydroxybenzoate, benzyl p-hydroxybenzoate, p-hydroxybenzoate-p-chlorobenzyl, p-hydroxybenzoate-o-chlorobenzyl, p-hydroxybenzoate-p-methylbenzyl, p-hydroxybenzoate-n-octyl, benzoic acid, zinc salicylate, 1-hydroxy-2-naphthoic acid, 2-hydroxy-6-naphthoic acid, 2-hydroxy-6-zinc naphthoate, 4-hydroxy diphenyl sulphone, 4-hydroxy-4′-chloro diphenyl sulfone, bis (4-hydroxy phenyl) sulfide, 2-hydroxy-p-toluic acid, 3,5-di-tert-zinc butyl salicylate, 3,5-di-tert-tin butyl salicylate, tartaric acid, oxalic acid, maleic acid, citric acid, succinic acid, stearic acid, 4-hydroxyphthalic acid, boric acid, thiourea derivative, 4-hydroxy thiophenol derivative, bis(4-hydroxyphenyl) acetate, bis(4-hydroxyphenyl)ethyl acetate, bis (4-hydroxyphenyl acetate-n-propyl, bis (4-hydroxyphenyl) acetate-n-butyl, bis (4-hydroxyphenyl) phenyl acetate, bis (4-hydroxyphenyl) benzyl acetate, bis (4-hydroxyphenyl) phenethyl acetate, bis (3-methyl-4-hydroxyphenyl) acetate, bis(3-methyl-4-hydroxyphenyl) methyl acetate, bis (3-methyl-4-hydroxyphenyl) acetate-n-propyl, 1,7-bis(4-hydroxyphenylthio) 3,5-dioxaheptane, 1,5-bis(4-hydroxyphenylthio) 3-oxaheptane, 4-hydroxy phthalate dimethyl, 4-hydroxy-4′-methoxy phenyl sulfone, 4-hydroxy-4′-ethoxy diphenyl sulfone, 4-hydroxy-4′-isopropoxy diphenyl sulfone, 4-hydroxy-4′-propoxy diphenyl sulfone, 4-hydroxy-4′-butoxy diphenyl sulfone, 4-hydroxy-4′-isopropoxy diphenyl sulfone, 4-hydroxy-4′-sec-butoxy diphenyl sulfone, 4-hydroxy-4′-tert-butoxy diphenyl sulfone, 4-hydroxy-4′-benzyloxy diphenyl sulfone, 4-hydroxy-4′-phenoxy diphenyl sulfone, 4-hydroxy-4′-(m-methyl benzoxy) diphenyl sulfone, 4-hydroxy-4′-(p-methyl benzoxy) diphenyl sulfone, 4-hydroxy-4′-(o-methyl benzoxy) diphenyl sulfone, 4-hydroxy-4′-(p-chloro benzoxy) diphenyl sulfone, 4-hydroxy-4′-oxyaryl diphenyl sulfone and the like. The sensitive thermochromic dye may be bisphenol A. The temperature sensitive thermochromic dye may be from 0.01 to 10 wt %.

In one embodiment the present invention provides a method of preventing cell and/or tissue damage and loss of the implant by (a) providing a thermochromic dental composition comprising: a polymeric dental composition; and a temperature sensitive thermochromic dye in contact with the polymeric dental composition, wherein the temperature sensitive thermochromic dye is colorless at body temperature and undergoes a reversible color change at a temperature of between 99-160° F.; (b) placing the thermochromic dental composition in a patient; (c) modifying the thermochromic dental composition, wherein heat is generated in the thermochromic dental composition and the temperature sensitive thermochromic dye changes color at a temperature; (d) allowing the thermochromic dental composition to cool to below the temperature; and (e) repeating (c) and (d).

In one embodiment the present invention provides a thermochromic composite material comprising: a polymeric dental composition; and a temperature sensitive thermochromic dye in contact with the polymeric dental composition, wherein the temperature sensitive thermochromic dye is colorless at body temperature and undergoes a reversible color change at a temperature of between 99-160° F.

In one embodiment the present invention provides a temperature indicating dental material comprising a polymeric dental composition; and a temperature sensitive thermochromic dye in contact with the polymeric dental composition, wherein the temperature sensitive thermochromic dye is colorless at body temperature and undergoes a reversible color change at a temperature of between 99-160° F.

In one embodiment the present invention provides a temperature indicating abutment comprising: a polymeric dental composition; and a temperature sensitive thermochromic dye in contact with the polymeric dental composition, wherein the temperature sensitive thermochromic dye is colorless at body temperature and undergoes a reversible color change at a temperature of between 99-160° F.

In one embodiment the present invention provides a temperature indicating reduction coping comprising: a polymeric dental composition; and a temperature sensitive thermochromic dye in contact with the polymeric dental composition, wherein the temperature sensitive thermochromic dye is colorless at body temperature and undergoes a reversible color change at a temperature of between 99-160° F.

A method of making a temperature indicating dental material by providing one or more polymerizable monomers for use in a dental composition; mixing one or more temperature sensitive thermochromic dye with the one or more polymerizable monomers; forming a dental composition; and curing the dental composition, wherein the temperature sensitive thermochromic dye is colorless at body temperature and undergoes a reversible color change at a temperature of between 99-160° F.

A method of making a thermochromic abutment by providing one or more polymerizable monomers for use in a dental composition; mixing one or more temperature sensitive thermochromic dye with the one or more polymerizable monomers; forming a dental composition; and curing the dental composition, wherein the temperature sensitive thermochromic dye is colorless at body temperature and undergoes a reversible color change at a temperature of between 99-160° F.

A method of fitting a thermochromic abutment by (a) providing a thermochromic dental composition comprising: a polymeric dental composition; and a temperature sensitive thermochromic dye in contact with the polymeric dental composition, wherein the temperature sensitive thermochromic dye is colorless at body temperature and undergoes a reversible color change at a temperature of between 99-160° F.; (b) placing the thermochromic dental composition in a patient; (c) modifying the thermochromic dental composition, wherein heat is generated in the thermochromic dental composition and the temperature sensitive thermochromic dye changes color at a temperature; (d) allowing the thermochromic dental composition to cool to below the temperature; and (e) repeating (c) and (d).

### BRIEF DESCRIPTION OF THE DRAWINGS

None.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, the term “dental materials” means materials which are suitable for use in the patient's mouth, i.e. for example as a dental restoration, as a constituent of a dental restoration or serve to fix a dental restoration, orthopaedic or prosthetic device in the mouth and include but are not limited to prosthetics, filling materials, cements for inlays, onlays, bridges, crowns, implants, screws, pegs, pins, natural tooth, bones, surfaces, coatings, filling materials, adhesives, cements or other substance.

As used herein, the terms “thermochromic dyes” and “color changing component” are used interchangeably to mean inorganic or organic substances which reversibly change their color according to their temperature.

The present invention relates to implant restoration. After a dental implant is placed in the bone it must be restored with a fixed or removable prosthetic to be useful to the patient. The fixed prosthetic can be a single crown, multiple unit bridge, or bar secured to the implants to stabilize the prosthetic restoration. The removable prosthetic can be a full or partial denture. Once the implant has been successfully placed the portion of the implant that is seen above the gum line is usually referred to as the abutment portion. The abutment can be a separate piece that is screwed into the portion of the implant that is beneath the gum and in the bone (e.g., a two piece implant) or it can be the portion of a one piece implant that is above the gum line. The implant abutment has a specific shape that allows for a restoration to be placed over it and taken on and off before cementation or being screwed into place. This shape is tapered slightly and allows the restoration to draw. On many occasions the implant abutment needs to be further shaped or prepared beyond the original shape to create the ideal form required in each unique situation. If the abutment is shaped in the mouth as required of a one piece implant or as elected with a two piece it is usually done with the standard air driven hand piece and carbide burr. The two piece abutment is sometimes “picked up” in an impression technique and prepared on a model in the lab. This adds to the cost of the procedure as labs charge for this preparation. Whenever the abutment is shaped or prepared in the mouth heat is generated as the carbide bur turns at 50,000 or more rpm. For that reason some implant manufacturers recommend that no preparation is done in the mouth. However, a one piece implant cannot be prepared in the lab and must be prepared in the mouth. As a result, dentists have no indication or assurance that excessive heating the implant is occurring and therefore excessive heating of the bone and surrounding tissue. Usually copious irrigation with water is used to cool the implant and surrounding tissue to try to avoid overheating the implant and tissue damage or death.

The present invention relates to a visual indicator of the excessive heating the implant using a thermochromic dental material applied to the implant abutment prior to preparation or made as a part of the implant abutment. The materials will change color once a temperature is achieved and return to their original color once the temperature returns to below the threshold. The present invention is a valuable addition to implant treatment process by giving dentists a method to measure the temperature of the implant and to adjust their efforts to avoid excessive heating the implant. The thermochromic materials are accurate to within one degree F. and the target temperature can be specified during the manufacturing of the thermochromic material. The thermochromic material can be made of FDA approved materials that are nontoxic as with those that are used for the manufacturing of plastic spoons used for infants to prevent excessively hot food from touching the infants tissues.

The thermochromic material can be mixed in a dental composite, dental acrylic, polymer, or a plastic type material that can be placed on the abutment before the abutment is prepared. It can be applied directly to the two piece metal abutment in manufacturing or the abutment portion of the implant during manufacturing.

When mixed in a composite material. The composite is applied to the abutment and then cured with UV light until it sets and becomes a hard skin over the abutment that has an intimate contact and serves as a thin veneer over the abutment. When the abutment is prepared and heat is generated that exceeds the desired temperature then the composite changes color notifying the dentist to stop preparation for a few seconds until the temperature is restored to a safe level that will not cause cellular damage or death. In this way the dentist is assured that there is no damage to the bone that might result in loss of the implant. The present invention provides a safe method to prepare an implant abutment as well as a method that gives the dentist confidence and control of the process in a way that is not available in the current practice of dentistry.

Another embodiment includes the incorporation into what is known as a reduction coping. When the lab has the models of the mouth they are able to evaluate if there is the exact amount of space, angle, or vertical clearance necessary to fabricate the prosthetic for the patient. A reduction coping is sometimes produced by the lab so that the dentist can place the coping on the implant in the mouth and prepare or cut off the portion of the implant abutment that that extends out or beyond the reduction coping. At this time the reduction coping is usually fabricated out of metal and the dentist places the metal coping over the abutment and cuts off the amount of the abutment down to the edges of the abutment coping. The reduction coping can be fabricated out of any material that is used in dentistry and the thermochromic material can be incorporated in the material or if the coping is made of metal it can be applied to the metal.

The thermochromic material can be incorporated in any material that can be applied to the abutment. Some of the materials are a composite that can be painted on and hardened in the mouth, or a prefabricated composite coping that that can be placed over the abutment and serve as a substructure for the fabrication of a temporary crown or restoration. The temporary will later be removed my cutting it off. The material can be placed in composites or acrylics or plastics to be used as a reduction coping. These are some of the uses of the thermochromic material to prevent overheating of the abutment and the possible result of cell damage and loss of the implant.

The '436 Patent discloses a thermochromic dental material including a low temperature composition that is used for the purpose of distinguished from the dental material from natural tooth substances. Tooth-colored restoration materials have the disadvantage that they can be visually distinguished from the natural tooth substance with difficulty and results in removal of excess material and not removing all of the material making the reworking and matching difficult. The '436 Patent discloses thermochromic dyes that are colorless at a temperature of approximately 37° C. and which change color upon heating or preferably cooling, i.e. assume a color that can clearly be distinguished from the natural tooth substance. At a temperature of about 37° C. the color of the dental material is thus determined by its intrinsic color but change color at a temperature of 84° C. or less, preferably of 41 to 84° C. The materials '436 Patent discloses thermochromic dyes which change color at temperatures of 104 to 140° C. and in particular 113 to 131° C. However, the '436 Patent relates to the cooling of the thermochromic dyes as an indicator of the presence of the dental material.

In contrast to dental materials currently in use and the thermochromic dye of the '436 Patent, the present invention provides a thermochromic dental material that temporarily changes color as a function of elevated temperature to indicate an elevated temperature at the dental material and potential damage to the prosthetic and surrounding tissues (e.g., cell death) from the heat. At the elevated temperatures the dental material can be visually distinguished from the natural tooth and visually indicates an increased temperature at the dental material and at the surrounding tissue as a result of heat transfer. At elevated temperatures the heat transfer to the surrounding tissues can be damaged to the point of cell death. The thermochromic dye becomes a visible indication of impending danger to the surrounding tissues and that the dental material should be allowed to cool. Once the temperature of the dental material has receded to a temperature that is below the thermochromic transition temperature of the thermochromic dye the dental material again assumes its original color. The dental material can then be after a period sufficient for the working of the dental material.

In an embodiment of the present invention, the dental material includes one thermochromic dye that indicates that the temperature is at or above a preselected temperature. In another embodiment, the dental material includes more than one thermochromic dye that indicates that the temperature is at or above a preselected temperature to provide sequential, progressive and increasing warnings as to the increasing risk of physical harm to the tissue and/or prosthetic. For example, the dental material may appear to be normal colored at ordinary body temperature and as the temperature increases the color of the dental material changes. The dental material may indicate that the temperature is at or above 100, 101, 102, 103, 104, 105, 110, 115, 120, 125, 130, 135, 140, 145, 150, 155° F. or incremental variations thereof. In use, a prosthetic is made from a dental material containing a thermochromic dye and is a natural color but as the prosthetic is heated (e.g., through physical manipulation of the prosthetic or heating by an external source) the thermochromic dye indicates that the temperature is at or above 99, 100, 101, 102, 103, 104, 105, 110, 115, 120, 125, 130, 135, 140, 145, 150, 155° F. For example the present the prosthetic including the thermochromic dye is a natural color at normal bodily temperatures when positioned in the mouth. As the prosthetic is heated (e.g., through physical manipulation) the thermochromic dye changes color to indicate the increase in temperature. As the prosthetic is cooled the thermochromic dye reverts to the natural color. When the prosthetic is re-heated the thermochromic dye again changes color to indicate the increase in temperature and again as the prosthetic is cooled the thermochromic dye reverts to the natural color. In addition the prosthetic may include numerous thermochromic dyes at a natural color at normal bodily temperatures when positioned in the mouth and change to different colors as the prosthetic is heated to indicate the increase in temperature. The color may be any color, e.g., white, pink, red, orange, brown, yellow, gray, green, cyan, blue, violet, and incremental shades thereof.

In another use, the prosthetic includes a dental material having 2 or more dental materials in the form of a prosthetic having a natural color at bodily temperatures. As the prosthetic is heated the first color changing component indicates that the temperature is at or above a first temperature. As the temperature continues to increase a second color changing component indicates that the temperature is at or above a second temperature. This process can be continued through numerous temperatures and ranges. As the temperature of the prosthetic approaches a dangerous level, the color changing component provides a visible indication of the temperature level and potential damage to tissue as a result.

In one embodiment, the preselected temperature of color change of the element is selected to show that the temperature of prosthetic is between about 99 and 175° F. In one embodiment, the preselected temperature of color change of the element is selected to show that the temperature of prosthetic is at or above 105° F. In another particularly embodiment, the preselected temperature of color change of the element is selected to show that the temperature of prosthetic is at or above 110° F. In yet another particularly embodiment, the preselected temperature of color change is selected to show that the temperature of the prosthetic is at or above 115° F. In another particularly embodiment, the preselected temperature of color change of the element is selected to show that the temperature of the prosthetic is at or above 120° F. In another particularly embodiment, the preselected temperature of color change of the element is selected to show that the temperature of prosthetic is at or above 130° F. In another particularly embodiment, the preselected temperature of color change of the element is selected to show that the temperature of prosthetic is at or above 140° F. In another particularly embodiment, the preselected temperature of color change of the element is selected to show that the temperature of prosthetic is at or above 150° F. In another particularly embodiment, the preselected temperature of color change of the element is selected to show that the temperature of prosthetic is at or above 160° F. These preselected temperature levels correspond to body temperatures that represent various degrees of danger in increasing body temperature, wherein the risk of physical harm to tissue increases with increasing temperature.

The thermochromic dye can be any composition that is sensitive to a change in temperature having a first color at a first temperature and a second color at a second temperature. In many instances the first color is a non-distinguishing color that is not visible at bodily temperatures but undergoes a transition and color change as the temperature exceeds a specific temperature.

One example of a thermochromic dye is a liquid crystal material, an electron donor and an electron acceptor pair, an acidic component and an acid-responsive component. Liquid crystal material include liquid crystalline cholesterol derivatives, such as alkanic acid and aralkanic acid esters of cholesterol, alkyl esters of cholesterol carbonate and mixtures thereof, in particular those with alkyl and alkanic acid groups with 1 to 24 carbon atoms. Cholesterol esters and derivatives thereof which contain an alkanic acid group with 9 to 22 carbon atoms or an aralkanic acid group with a benzoic acid group and 1 to 3 carbon atoms in the alkyl part are particularly preferred. In the case of the cholesterol carbonate esters, those with C₁ to C₂₀ alkyl groups are preferred. Further liquid-crystalline cholesterol derivatives include cholesterol chloride, cholesterol bromide, cholesterol acetate, cholesterol oleate, cholesterol caprylate, cholesterol oleyl-carbonate, mixtures thereof and mixtures of these dyes with the previously named cholesterol derivatives.

The thermochromic dyes include an electron donors may include substituted phenyl-methanes, fluoranes, such as for example 3,3′-dimethoxy-fluorane, 3-chloro-6-phenylamino-flourane, 3-diethylamino-6-methyl-7-chlorofluorane, 3-diethyl-7,8-benzofluorane, 3,3′, 3″-tris(p-di-methylaminophenyl)phthalide, 3,3′-bis(p- dimethyl-aminophenyl)-7-phenylaminofluorane and 3-diethyl-amino-6-methyl-7-phenylamino-fluorane, indolylphthalides, spiropy-ranes and cumarins, as well as mixtures of these substances. Electron acceptors include phenols, azoles, organic acids and esters as well as salts of organic acids. Examples that may be cited as phenols are phenylphenol, bisphenol A, cresol, resorcinol, chlorolucinol, phenol, phenol oligomers, β-naphthol, 1,5-dihydroxynaphthalene, pyrocatechol, pyrogallol, and the trimer of p-chlorophenol formaldehyde condensate. Examples that may be cited as azoles are benzo-triazoles, such as 5-chlorobenzo-triazole, 4-laurylaminosulfo-benzotriazole, 5-butylbenzotriazole, dibenzotrizaole, 2-oxy-benzotriazole, 5-ethoxy-carbonylbenzo-triazole, 5,5′-methylene-bisbenzotriazole, imidazole, such as oxybenzimidazole, and tetrazole. The organic acids comprise for example aromatic and aliphatic carboxylic acids and substituted derivatives thereof. Examples of aromatic carboxylic acids are salicylic acid, methylenebissalicylic acid, β-resorcylic acid, gallic acid, benzoic acid, p-oxy-benzoic acid, pyromellitic acid, β-naphthoic acid, tannic acid, toluic acid, trimellitic acid, phthalic acid, terephthalic acid and anthranalic acid. Examples of aliphatic carboxylic acids are acids with 1 to 20 carbon atoms, preferably 3 to 15 carbon atoms, such as for example stearic acid, 1,2-hydroxystearic acid, tartaric acid, citric acid, oxalic acid and lauric acid. Examples of esters are alkyl esters of aromatic carboxylic acids in which the alkyl group contains 1 to 6 carbon atoms, such as butyl gallate, ethyl-p-hydroxybenzoate and methyl salicylate. Examples that may be cited as salts are ammonium and metal salts of the above-named acids. The metal salts comprise for example lithium, sodium, calcium, magnesium, aluminum, zinc, tin, titanium and nickel salts. Particularly electron acceptors are 1,2-hydroxystearic acid, tartaric acid and citric acid. The above-named electron acceptors can be used alone or mixed with one another. Furthermore, the thermochromic materials can be used as such or in microencapsulated form.

The thermochromic dyes include an acid-responsive component and an acidic component mixture or an acid-responsive chromogenic substance and an acidic substance (acid component). The acid-responsive substances include triphenylmethane phthalides, phthalides, phthalanes, acyl-leucomethylene blue compounds, fluoranes, triphenylmethanes, diphenylmethanes, spiropyranes and derivatives of these substances. Exemplary compounds are 3,6-dimethoxyfluorane , 3,6-di-butoxyfluorane, 3-diethylamino-6,-dimethylfluorane, 3 -chloro-6-phenylamino-fluorane, 3-diethylamino-6-methyl-7-chloro-fluorane, 3-diethyl-amino-7,8-benzofluorane, 2-anilino-3-methyl-6-diethylamino-fluorane, 3,3′, 3″-tris(p-dimethylamino-phenyl)phthalide, 3,3′-bis(p-dimethyl-aminophenyl)phthalide, 3-diethylamino-7-phenyl-aminofluorane, 3,3-bis(p-diethylamino-phenyl)-6-dimethylamino-phthalide, 3-(4-diethylaminophenyl)-3-(1-ethyl-2-methylindo1-3 -yl)phthalide,3-(4-diet hylamino-2-methyl)phenyl-3-(1,2-dimethylindo1-3-yl)phthalide and 2′-(2-chloranilino)-6′-dibutylamino-spiro-[phthalido-3,9′-xanthene]. The acidic substances include 1,2,3-benzotriazoles, phenols, thioureas, oxoaromatic carboxylic acids and derivatives of these substances. Exemplary compounds are 5-butylbenzotriazole, bisbenzotrizaol-5-methane, phenol, nonylphenol, bisphenol A, bisphenol F, 2,2′-biphenol, β-naphthol, 1,5-dihydroxynaphthalene, alkyl-p-hydroxybenzoates and phenolic resin oligomers. These dyes can likewise be used as such or in micro-encapsulated form.

The thermochromic compositions or dyes can be thermochromic powders or thermochromic micro capsules in a powder pigment format, thermochromic slurries or thermochromic micro capsules in an aqueous based dispersion form, thermochromic inks or thermochromic masterbatch comprises of thermochromic blended with a polymer masterbatch carrier and come in pellet form. Examples of the thermochromic compositions can be purchased from numerous suppliers (e.g., QCR Solutions Corp) and may be fine-tuned to a specific temperature. For example, thermochromic leuco dyes may be used in colors include black, blue, magenta, green, orange, red, purple, brown and custom matched colors. In addition the activation temperatures of the thermochromic dye can be set anywhere between 99° F. through 160° F. as desired by the specific application.

The thermochromic dye can be selected from a group consisting of a thermocromatic ink, a thermocromatic liquid crystal ink, a chiro nematic liquid crystal ink, cholesteryl nonanoate, a cyanobisphenyl, a combination of sholesteryl and nematic inks, a mixture of leuco dyes with a weak acid, a mixture of crystal violet lactone, a spirolactone, a fluoran, a spiropyran, a fulgide or a fulgide with bisphenol A, a paraben, octadecylphosphoric acid, a 1,2,3-triazole derivative or 4-hydroxycoumarin, a mineral oxide, zinc oxide, lead oxide, cuprous mercury iodide, mercury iodide, nickel sulfate, a chromium rich pyrope, a chrystalline bismuth oxychloride, an organosiloxane containing methacryloyl or acryloyl functional groups, an organosiloxane containing nuclei that are divalent radicals consisting of 1,4 phenylene, 1,4 cyclohexylene, 2,2 pyridinylene, 2,5 pyranylene, 5,2 pyrimidinylene, 2,5-(1,3-dioxanylene), 2,6-naphthylidene and 1,4-naphylidene and combinations thereof.

The reversible thermochromic dye material requires a thermosensitive capability to repeat color development and color erasure. For example, the present invention includes an electron-donating color-forming compound, namely a dye precursor (leuco dye) which is colorless or pale. The thermochromic dye is not particularly limited, and examples thereof include fluoran compounds, triphenylmethane phthalide compounds, azaphthalide compounds, phenothiazine compounds, leucoauramine compounds and indolinophthalide compounds.

Other examples of the thermochromic dye include 2-anilino-3-methyl-6-diethylaminofluoran, 2-anilino-3-methyl-6-di(n-butylamino)fluoran, 2-anilino-3-methyl-6-(N-n-propyl-N-methylamino)fluoran, 2-anilino-3-methyl-6-(N-isopropyl-N-methylamino)fluoran, 2-anilino-3-methyl-6-(N-isobutyl-N-methylamino)fluoran, 2-anilino-3-methyl-6-(N-n-amyl-N-methylamino)fluoran, 2-anilino-3-methyl-6-(N-sec-butyl-N-methylamino)fluoran, 2-anilino-3-methyl-6-(N-n-amyl-N-ethylamino)fluoran, 2-anilino-3-methyl-6-(N-iso-amyl-N-ethylamino)fluoran, 2-anilino-3-methyl-6-(N-n-propyl-N-isopropylamino)fluoran, 2-anilino-3-methyl-6-(N-cyclohexyl-N-methylamino)fluoran, 2-anilino-3-methyl-6-(N-ethyl-p-toluidino)fluoran, 2-anilino-3-methyl-6-(N-methyl-p-toluidino)fluoran, 2-(m-trichloromethylanilino)-3 -methyl-6-diethylaminofluoran, 2-(m-trifluoromethylanilino)-3-methyl-6-diethylaminofluoran, 2-(m-trichloromethylanilino)-3 -methyl-6-(N-cyclohexyl-N-methylamino)fluoran, 2-(2,4-dimethylanilino)-3-methyl-6-diethylaminofluoran, 2-(N-ethyl-p-toluidino)-3 -methyl-6-(N-ethylanilino)fluoran, 2-(N- ethyl-p-toluidino)-3 -methyl-6-(N-propyl-p-toluidino)fluoran, 2-anilino-6-(N-n-hexyl-N-ethylamino)fluoran, 2-(o-chloroanilino)-6-diethylaminofluoran, 2-(o-chloroanilino)-6-dibutylaminofluoran, 2-(m-trifluoromethylanilino)-6-diethylaminofluoran, 2,3-dimethyl-6-dimethylaminofluoran, 3-methyl-6-(N- ethyl-p-toluidino)fluoran, 2-chloro-6-diethylaminofluoran, 2-bromo-6-diethylaminofluoran, 2-chloro-6-dipropylaminofluoran, 3-chloro-6-cyclohexylamino fluoran, 3-bromo-6-cyclohexylaminofluoran, 2-chloro-6-(N-ethyl-N-isoamylamino)fluoran, 2-chloro-3-methyl-6-diethylaminofluoran, 2-anilino-3-chloro-6-diethylaminofluoran, 2-(o-chloroanilino)-3-chloro-6-cyclohexylaminofluoran, 2-(m-trifluoromethylanilino)-3-chloro-6-diethylaminofluoran, 2-(2,3-dichloroanilino)-3-chloro-6-diethylaminofluoran, 1,2-benzo-6-diethylaminofluoran, 3-diethylamino-6-(m-trifluoromethylanilino)fluoran, 3-(1- ethyl-2-methylindole-3-yl)-3-(2-ethoxy-4-diethylaminophenyl)-4-azaph-thalide, 3-(1-ethyl-2-methylindole-3-yl)-3-(2-ethoxy-4-diethylaminophenyl)-7-azaphthalide, 3-(1-octyl-2-methylindole-3-yl)-3-(2- ethoxy-4-diethylaminophenyl)-4-azaph-thalide, 3-(1-ethyl-2-methylindole-3-yl)-3-(2-methyl-4-diethylaminophenyl)-4-azaphthalide, 3-(1-ethyl-2-methylindole-3-yl)-3-(2-methyl-4-diethylaminophenyl)-7-azaph-thalide, 3-(1-ethyl-2-methylindole-3-yl)-3 -(4-diethylaminophenyl)-4-azapht-halide, 3-(1- ethyl-2-methylindole-3-yl)-3-(4-N-n-amyl-N-methylaminophenyl)-4-azaphthalide, 3-(1-methyl-2-methylindole-3-yl)-3-(2-hexyloxy-4-diethylaminophenyl)-4-azaphthalide, 3,3-bis(2-ethoxy-4-diethylaminophenyl)-4-azaphthalide and 3,3-bis(2-ethoxy-4-diethylaminophenyl)-7-azaphthalide.

The thermochromic dye used in the present invention may also be selected from conventionally known leuco dyes including 2-(p-acetylanilino)-6-(N-n-amyl-N-n-butylamino)fluoran, 2-benzylamino-6-(N-ethyl-p-toluidino)fluoran, 2-benzylamino-6-(N-methyl-2,4-dimethylanilino)fluoran, 2-benzylamino-6-(N-ethyl-2,4-dimethylanilino)fluoran, 2-benzylamino-6-(N-methyl-p-toluidino)fluoran, 2-benzylamino-6-(N-ethyl-p-toluidino)fluoran, 2-(di-p-methylbenzylamino)-6-(N-ethyl-p-toluidino)fluoran, 2-(α-phenylethylamino)-6-(N-ethyl-p-toluidino)fluoran, 2-methylamino-6-(N-methylanilino)fluoran, 2-methylamino-6-(N-ethylanilino)fluoran, 2-methylamino-6-(N-propylanilino)fluoran, 2-ethylamino-6-(N-methyl-p-toluidino)fluoran, 2-methylamino-6-(N-methyl-2,4-dimethylanilino)fluoran, 2-ethylamino-6-(N-ethyl-2,4-dimethylanilino)fluoran, 2-dimethylamino-6-(N-methylanilino)fluoran, 2-dimethylamino-6-(N-ethylanilino)fluoran, 2-diethylamino-6-(N-methyl-p-toluidino)fluoran, 2-diethylamino-6-(N-ethyl-p-toluidino)fluoran, 2-dipropylamino-6-(N-methylanilino)fluoran, 2-dipropylamino-6-(N-ethylanilino)fluoran, 2-amino-6-(N-methylanilino)fluoran, 2-amino-6-(N-ethylanilino)fluoran, 2-amino-6-(N-propylanilino)fluoran, 2-amino-6-(N-methyl-p-toluidino)fluoran, 2-amino-6-(N-ethyl-p-toluidino)fluoran, 2-amino-6-(N-propyl-p-toluidino)fluoran, 2-amino-6-(N-methyl-p-ethylanilino)fluoran, 2-amino-6-(N-ethyl-p-ethylanilino)fluoran, 2-amino-6-(N-propyl-p-ethylanilino)fluoran, 2-amino-6-(N-methyl-2,4-dimethylanilino)fluoran, 2-amino-6-(N-ethyl-2,4-dimethylanilino)fluoran, 2-amino-6-(N-propyl-2,4-dimethylanilino)fluoran, 2-amino-6-(N-methyl-p-chloroanilino)fluoran, 2-amino-6-(N-ethyl-p-chloroanilino)fluoran, 2-amino-6-(N-propyl-p-chloroanilino)fluoran, 1,2-benzo-6-(N-ethyl-N-isoamylamino)fluoran, 1,2-benzo-6-dibutylaminofluoran, 1,2-benzo-6-(N-methyl-N-cyclohexylamino)fluoran and 1,2-benzo-6-(N-ethyl-N-toluidino)fluoran. Each of these may be used alone or in combination.

The present invention also uses any compound that is an electron-accepting compound and has a function of making the thermochromic dye develop color. Examples of conventionally known compositions include organic phosphoric acid compounds, aliphatic carboxylic acid compounds, phenol compounds, and metal salts and phosphates of mercaptoacetic acid. The composition may be dispersed along with the thermochromic dye, with addition of a dispersant and/or a surfactant. The thermochromic dyes are preferably used in a quantity of between 0.01 to 2 wt. %, particularly preferably between 0.01 to 25 wt. % or between 0.1 to 0.5 wt. % or approx. 0.2 wt. % relative to the total mass of the dental material.

The thermochromic dyes may include but not limited to leuco dyes include bisphenol A, tetrabromobisphenol A, gallnut acid, salicylic acid, 3-isopropyl salicylate, 3-cyclohexyl salicylate, 3-5-di-tert-butyl salicylate, 3,5-di-α-methyl benzyl salicylate, 4,4′-isopropylidenediphenol, 1,1′-isopropylidene bis (2-chlorophenol), 4,4′-isopropylene bis (2,6-dibromophenol), 4,4′-isopropylidene bis (2,6-dichlorophenol), 4,4′-isopropylidene bis(2-ethyl phenol), 4,4′-isopropylidene bis(2,6-dimethyl phenol), 4,4′-isopropylidene bis (2-tert-butyl phenol), 4,4′-sec-butylidene diphenol, 4,4′-cyclohexylidene bisphenol, 4,4′-cyclohexylidene bis (2-ethyl phenol), 4-tert-butyl phenol, 4-phenyl phenol, 4-hydroxy diphenoxide, α-naphthol, (β-naphthol, 3,5-xylenol, thymol, methyl-4-hydroxybenzoate, 4-hydroxyacetophenone, novolak phenol resins, 2,2′-thio bis(4,6-dichloro phenol), catechol, resorcin, hydroxynone, hydroquinone, pyrogallol, fluoroglycine, fluoroglycine carbonate, 4-tert-octyl catechol, 2,2′-methylene bis (4-chlorophenol), 2,2′-methylene bis (4-methyl-6-tert-butyl phenol), 2,2′-dihydroxy diphenyl, ethyl p-hydroxybenzoate, propyl p-hydroxybenzoate, butyl p-hydroxybenzoate, benzyl p-hydroxybenzoate, p-hydroxybenzoate-p-chlorobenzyl, p-hydroxybenzoate-o-chlorobenzyl, p-hydroxybenzoate-p-methylbenzyl, p-hydroxybenzoate-n-octyl, benzoic acid, zinc salicylate, 1-hydroxy-2-naphthoic acid, 2-hydroxy-6-naphthoic acid, 2-hydroxy-6-zinc naphthoate, 4-hydroxy diphenyl sulphone, 4-hydroxy-4′-chloro diphenyl sulfone, bis (4-hydroxy phenyl) sulfide, 2-hydroxy-p-toluic acid, 3,5-di-tert-zinc butyl salicylate, 3,5-di-tert-tin butyl salicylate, tartaric acid, oxalic acid, maleic acid, citric acid, succinic acid, stearic acid, 4-hydroxyphthalic acid, boric acid, thiourea derivative, 4-hydroxy thiophenol derivative, bis(4-hydroxyphenyl) acetate, bis(4-hydroxyphenyl)ethyl acetate, bis (4-hydroxyphenyl acetate-n-propyl, bis (4-hydroxyphenyl) acetate-n-butyl, bis (4-hydroxyphenyl) phenyl acetate, bis (4-hydroxyphenyl) benzyl acetate, bis (4-hydroxyphenyl) phenethyl acetate, bis (3-methyl-4-hydroxyphenyl) acetate, bis(3-methyl-4-hydroxyphenyl) methyl acetate, bis (3-methyl-4-hydroxyphenyl) acetate-n-propyl, 1,7-bis(4-hydroxyphenylthio) 3,5-dioxaheptane, 1,5-bis(4-hydroxyphenylthio) 3-oxaheptane, 4-hydroxy phthalate dimethyl, 4-hydroxy-4′-methoxy phenyl sulfone, 4-hydroxy-4′-ethoxy diphenyl sulfone, 4-hydroxy-4′-isopropoxy diphenyl sulfone, 4-hydroxy-4′-propoxy diphenyl sulfone, 4-hydroxy-4′-butoxy diphenyl sulfone, 4-hydroxy-4′-isopropoxy diphenyl sulfone, 4-hydroxy-4′-sec-butoxy diphenyl sulfone, 4-hydroxy-4′-tert-butoxy diphenyl sulfone, 4-hydroxy-4′-benzyloxy diphenyl sulfone, 4-hydroxy-4′-phenoxy diphenyl sulfone, 4-hydroxy-4′-(m-methyl benzoxy) diphenyl sulfone, 4-hydroxy-4′-(p-methyl benzoxy) diphenyl sulfone, 4-hydroxy-4′-(o-methyl benzoxy) diphenyl sulfone, 4-hydroxy-4′-(p-chloro benzoxy) diphenyl sulfone, 4-hydroxy-4′-oxyaryl diphenyl sulfone and the like.

Aminodihydrophenazines, such as 3,7-bis(benzylethylamino)-5,10-dihydro-5-phenylphenazine, 3,7-bis(dimethylamino) -5-(p-chlorophenyl) -5,10-dihydrophenazine, 3,7-diamino-5,10-dihydro-5-methylphenazine, and 3,7-diamino-5,10-dihydro-2,5,8-trimethylphenazine. Aminodiphenylmethanes, such as 1,4-bis [bis (p-diethylaminophenyl) methyl] piperazine, bis (p-diethylaminophenyl)-1-benzotriazolylmethane, bis (p-diethylaminophenyl) (2,4-dichloroanilino) methane, bis(p-diethylaminophenyl) ( octadecylamino) methane, and 1,1-bis (p-dimethylaminophenyl)ethane. Aminohydrocinnamic acids (cyanoethanes), such as a-cyano-4-dimethylaminohydrocinnamamide, αβ-dicyano-4-dimethylaminohydrocinnamamide, αβ-dicyano-4-(p-chloroanilino) hydrocinnamic acid, methyl ester, p-(2,2-dicyanoethyl)-N,N-dimethylaniline, and 15p-(1,2,2-tricyanoethyl)-N,N-dimethylaniline. Leucoindigoid dyes, such as 7,7′-diamino-5,5′-dichloroleucothioindigo, 6,6′-dichloro-4-methylleucothioindigo, 7,7′-dimethylleucoindigo, 5,5′-disulfoleucoindigo, disodium salt, and 5,5′,7,7′-tetrachloroleucoindigo. 1,4-diamino-2,3-dihydroanthraquinones, such as 1,4-bis (ethylamino) -2,3-dihydroanthraquinone, 1-amino-4-methoxyanilino-2,3-dihydroanthraquinone, 1,4-diamino-2,3-dihydroanthraquinone, p-(2-hydroxyethyl amino) anilino-4-methylamino-2,3- dihydroanthraquinone. 1,4-bis(4,5-diaryl-2-imidazolyl) benzenes, such as 1,4-bis(4,5-diphenyl-2-imidazolyl) benzene, 1,4-bis [4,5-bis (p-methoxyphenyl) -2-imidazolyl] benzene, 1,4-bis [4,5-bis (o-chlorophenyl) -2-imidazolyl] benzene, 1,4-bis [4-(p-methoxyphenyl)-5-phenyl-2-imidazolyl] benzene. Hydroxyphenyldiarylimidazoles, such as 2-( p-hydroxyphenyl)-4,5 -diphenylimidazole, 2-(3,5-dibromo-4-hydroxyphenyl)-4,5-diphenylimidazole, 2-(3,5-dichloro-4-hydroxypheny 1)-4,5-diphenylimidazole, 2-(4-hydroxy-3,5-dimethoxyphenyl)-4,5-diphenylimidazole, 2-(3,5-dibromo-2-hydroxyphenyl)-4,5-diphenylimidazole, 4-(4-hydroxyphenyl) 2,5-diphenylimidazole, and 2-(4-hydroxy-3,5-dimethoxypheny 1)-4,5 -bis (pmethoxyphenyl) imidazole. Phthalic acid monoesters; Phthalic acid monobenzyl ester; Phthalic acid monocyclohexyl ester; Phthalic acid monophenyl ester; Phthalic acid monomethylphenyl ester; Phthalic acid monoethylphenyl ester; Phthalic acid monoalkylbenzyl ester; Phthalic acid monohalogenebenzyl ester; Phthalic acid monoalkoxybenzyl ester; Bis-(hydroxyphenyl)sulfides; Bis-(4-hydroxy-3-tert-butyl-6-methylphenyl)sulfide; Bis-(4-hydroxy-2,5-dimethylphenyl)sulfide; Bis-(4-hydroxy-2-methyl-5-ethylphenyl)1)sulfide; Bis-(4-hydroxy-2-methyl-5-isopropylphenyl)sulfide; Bis-(4-hydroxy-2,3-dimethylphenyl)sulfide; Bis-(4-hydroxy-2,5diethylphenyl)sulfide; Bis-(4-hydroxy 2,5-diisopropylphenyl)sulfide; Bis-(4-hydroxy-2,3,6-trimethylphenylsulfide; Bis-(2,4,5-trihydroxyphenyl)sulfide; Bis-(4-hydroxy-2-cyclohexyl-5-methylphenyl)sulfide; Bis-(2,3,4-trihydroxyphenyl)sulfide; Bis-(4,5-dihydroxy-2-tert-butylphenyl)sulfide; Bis-(4-hydroxy-2,5-diphenylphenyl)sulfide; Bis-(4-hydroxy-2-tert-o ctyl-5-methylphenyl)sulfide; 4-hydroxyphenylarylsulfones; 4-hydroxy-4′-isopropoxydiphenyl sulfone; 4-hydroxy-4′-methyldiphenyl sulfone; 4-hydroxy-4′-n-butyloxydiphenyl sulfone; 4-hydroxy phenylarylsulfonates; 4-hydroxyphenylbenzene sulfonate; 4-hydroxyphenyl-p-tolyl sulfonate; 4-hydroxyphenyl methylene sulfonate; 4-hydroxyphenyl-p-chlorobenzene sulfonate; 4-hydroxyphenyl-p-tert-butylbenzene sulfonate; 4-hydroxyphenyl-p-isopropoxybenzene sulfonate; 4-hydroxyphenyl-1′-naphthaline sulfonate; 4-hydroxyphenyl-2′-naphthaline sulfonate; 1,3-di[2-(hydroxyphenyl)-2-propyl]-benzenes; 1,3-di[2-(4-hydroxyphenyl)-2-propyl]-benzene; 1,3- di[2-(4-hydroxy-3-alkylphenyl)-2-propyl]-benzene; 1,3-di[2-(2,4 dihydroxyphenyl)-2-propyl]benzene; 1,3di[2 hydroxy-5-methylphenyl)-2-propyl]-benzene; Resorcinoles; 1,3-dihydroxy-6(α,αdimethylbenzyl)-benzene; 4-hydroxy benzoyloxybenzoic acid esters; 4-hydroxybenzoyloxybenzoic acid benzyl ester; 4-hydroxybenzoyloxybenzoic acid methyl ester; 4-hydroxybenzoyloxybenzoic acid ethyl ester; 4-hydroxybenzoyloxybenzoic acid propyl ester; 4hydroxybenzoyloxybenzoic acid butyl ester; 4-hydroxybenzoyloxybenzoic acid isopropyl ester; 4-hydroxybenzoyloxybenzoic acid tert.-butyl ester; 4-hydroxybenzoyloxybenzoic acid hexyl ester; 4-hydroxybenzoyloxybenzoic acid octyl ester; 4-hydroxybenzoyloxybenzoic acid nonyl ester; 4hydroxybenzoyloxybenzoic acid cyclohexyl ester; 4hydroxybenzoyloxybenzoic acid β-phenethyl ester; 4-hydroxybenzoyloxybenzoic acid phenyl ester; 4-hydroxybenzoyloxybenzoic acid α-naphthyl ester; 4-hydroxybenzoyloxybenzoic acid (β-naphthyl ester; 4-hydroxybenzoyloxybenzoic acid sec-butyl ester; bisphenol-sulfones (I); Bis-(3-1-butyl-4-hydroxy-6-methylphenyl)sulfone; Bis-(3-ethyl-4-hydroxyphenyl)sulfone; Bis-(3-propyl-4-hydroxyphenyl)sulfone; Bis-(3-methyl-4-hydroxyphenyl)sulfone; Bis-(2-isopropyl-4-hydroxyphenyl)sulfone; Bis-(2-ethyl-4-hydroxyphenyl)sulfone; Bis-(3-chloro-4-hydroxyphenyl)sulfone; Bis-(2,3-dimethyl-4-hydroxyphenyl)sulfone; Bis-(2,5-dimethyl-4-hydroxyphenyl)sulfone; Bis-(3-methoxy-4-hydroxyphenyl)sulfone; 4-hydroxyphenyl-2′-ethyl-4′-hydroxyphenylsulfone; 4-hydroxyphenyl-2′-isopropyl-4′-hydroxyphenylsulfone; 4-hydroxyphenyl-3′-isopropyl-4′-hydroxyphenylsulfone; 4-hydroxyphenyl-3′-sec-butyl-4′-hydroxyphenylsulfone; 3-chloro-4-hydroxyphenyl-3′-isopropyl4′-hydroxyphenylsulfone; 4hydroxy-5-t butylphenyl 4′-hydroxyphenylsulfone; 2hydroxy-5-t-amynophenyl-4,-hydroxyphenylsulfone; 2hydroxy-5-isopropylphenyl-4′-hydroxyphenylsulfone; 2hydroxy-5-t octylphenyl-4′-hydroxyphenylsulfone; 2hydroxy-5-t-butylphenyl 3′-chloro-4′-hydroxyphenylsulfone; 2hydroxy-5-t-butylphenyl-3′-methyl-4′-hydroxyphenylsulfone; 2hydroxy-5-t butylphenyl-3′-isopropyl-4′-hydroxyphenylsulfone; 2hydroxy-5-t-butylphenyl-3′-chloro-4′-hydroxyphenylsulfone; 2hydroxy 5-t-butylphenyl 3′-methyl-4′-hydroxyphenylsulfone; 2hydroxy-5-t-butylphenyl-3′-isopropyl-4′-hydroxyphenylsulfone; 2hydroxy-5-t-butylphenyl-2′-methyl-4′-hydroxyphenylsulfone; bisphenolsulfones (II); 4,4′-sulfonyldiphenol; 2,4′-sulfonyldiphenol; 3,3,- dichloro-4,4′-sulfonyldiphenol; 3,3′-dibromo-4,4′-sulfonyldiphenol; 3,3′,5,5′-tetrabromo-4,4′-sulfonyldiphenol; 3,3′diamino 4,4′-sulfonyldiphenol; p-tert-butylphenol; 2,4-hydroxybenzophenone; Novolac-phenolic resin; 4hydroxyacetophenone; p-phenylphenol; benzyl-4hydroxyphenyl acetate; and p-benzylphenol.

The thermochromic dyes may be added directly to the dental material, dispersed in a medium, dispersed in a binder, added to an epoxy or polymer, bound to a surface, added to an ink, pigment or coating. The color changing component may alternatively be placed within microcapsules. The microencapsulated component may be disbursed as such within the material of the dental material, or may be mixed with a binder in a dispersed condition. Additional components include additives, such as stabilizers, UV absorbers, polymerization inhibitors, dyes, pigments and lubricants.

One embodiment of the present invention includes dental materials including at least one polymerizable monomer, at least one initiator for photopolymerization and at least one thermochromic dye. The polymerizable monomer comprises an ethylene monomer, unsaturated ethylene monomer. Another composition includes amine-containing base paste with the peroxide-containing initiator paste distributed over two different components of the dental material and upon mixing radical polymerization is initiated by the reaction of amine and peroxide.

In addition to the thermochromic material, the dental materials according to the invention contain at least one ethylenically unsaturated monomer as binder and at least one initiator for hot, cold or preferably photopolymerization. In addition, the dental materials preferably also contain organic and/or inorganic filler.

There can be used, as initiators for photopolymerization, benzo-phenone and its derivatives for example, as well as benzoin and its derivatives. Further preferred photoinitiators are the α-diketones such as 9,10-phenanthrenequinone, diacetyl, furil, anisil, 4,4′-dichlorobenzil, 4,4′-dialkoxybenzil, phenylpropanedione and acylphosphine oxides. Camphorquinone is particularly preferably used. Photopolymerization is preferably initiated by irradiation with light in a wavelength range from 400 to 500 nm.

Suitable as polymerizable organic binders are all binders that can be used for a dental material, in particular monofunctional or polyfunctional methacrylates which can be used alone or in mixtures, e.g., binders are methyl methacrylate, isobutyl methacrylate, cyclohexyl methacrylate, tetraethylene glycol dimethacrylate, triethylene glycol dimethacrylate, diethylene glycol dimethacrylate, ethylene glycol dimethacrylate, polyethylene glycol dimeth-acrylate, butanediol dimethacrylate, hexanediol dimethacrylate, decanediol dimethacrylate, dodecanediol dimethacrylate, bis-phenol-A-dimethacrylate, trimethylolpropane trimethacrylate, 2,2-bis-[4-(2-hydroxy-3-methacryloxypropoxy)-phenyl]-propane(bis-GMA), as well as the reaction products of iscocyanates, in particular di- and/or triisocyanates and OH-group-containing methacrylates. Examples of these are the reaction products of 1 mol hexamethylene diisocyanate with 2 mol 2-hydroxyethylene methacrylate, of 1 mol tri-(6-isocyanatohexyl)bluret with 3 mol 2-hydroxyethyl methacrylate and of 1 mol 2,2,4-trimethyl-hexamethylene diisocyanate with 2 mol 2-hydroxyethyl methacrylate, which are called urethane dimethacrylates in the following. The proportion of these mostly long-chained compounds in the dental material varies between 10 and 80 wt. %. For example, 2,2-bis-[4-(2-hydroxy-3-meth-acryloxy-propoxy)-phenyl]-propane (bisphenol-A-diglycidyldimeth-acrylate, bis-GMA), 7,7,9-trimethyl-4,13 -dioxo-3,14-dioxa-5,12-diazahexa-decan-1,16-dioxy-dime thacrylate, triethylene glycol dimethacrylate, 1,10-decanediol dimethacrylate and mixtures of these monomers.

Initiators for the hot-curing systems include peroxides, in particular t-butyl peroxide, dibenzoyl peroxide, dilauroyl peroxide, tert.-butyl peroctoate and tert.-butyl perbenzoate. Moreover, 2,2′-azoisobutyronitrile (AIBN), benzopinacol and 2,2′-dialkybenzopinacols are also suitable. Hot-curing dental materials are particularly suitable for the production of inlays and onlays.

Used as initiators for cold polymerization are radical-supplying systems, for example benzoyl peroxide, lauroyl peroxide or preferably dibenzoyl peroxide, together with amines such as N,N-dimethyl-p-toluidine, N,N-dihydroxyethyl-p-toluidine or other structurally related amines.

To produce a light-curing filling material, 0.2% total mass of the dental material of the thermochromic dye were dispersed by means of a disperser in 21.1% of a monomer mixture of the composition given below. This mixture was then processed with 51.7% barium silicate glass powder, 5% barium fluorosilicate glass powder, 5% pyrogenic silica and 17% ytterbium trifluoride to produce a homogeneous composite. This was cured by three-minute irradiation with light of a wavelength of 400 to 500 nm. The cured material displayed a change of color at specific temperatures and after the temperature was lowered the color disappeared and the material again displayed its intrinsic color. This procedure was able to be repeated as often as wished.

Monomer mixture Bis-GMA 42.1% 7,7,9-trimethyl-4,13-dioxo-37% 3,14-dioxa-5,12-diazahexadecan-1,16-dioxy-dimethacrylate (diurethane dimethacrylate) Triethylene glycol dimethacrylate 20% Camphorquinone 0.3% Hydroquinone monoethyl ether 0.1% Ethyl-4-dimethylaminobenzoate 0.5% It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

What is claimed is:

1. A thermochromic dental material comprising:
a polymeric dental composition; and
a temperature sensitive thermochromic dye in contact with the polymeric dental composition, wherein the temperature sensitive thermochromic dye is colorless at body temperature and undergoes a reversible color change at a temperature of between 99-160° F.

2. The thermochromic dental material of claim 1, wherein the temperature sensitive thermochromic dye is a visual indicator a temperature of about 99, 100, 101, 102, 103, 105, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160° F., or above.

3. The thermochromic dental material of claim 1, wherein the temperature sensitive thermochromic dye is selected from bisphenol A, tetrabromobisphenol A, gallnut acid, salicylic acid, 3-isopropyl salicylate, 3-cyclohexyl salicylate, 3-5-di-tert-butyl salicylate, 3,5-di-α-methyl benzyl salicylate, 4,4′-isopropylidenediphenol, 1,1′-isopropylidene bis (2-chlorophenol), 4,4′-isopropylene bis (2,6-dibromophenol), 4,4′-isopropylidene bis (2,6-dichlorophenol), 4,4′-isopropylidene bis(2-ethyl phenol), 4,4′-isopropylidene bis(2,6-dimethyl phenol), 4,4′-isopropylidene bis (2-tert-butyl phenol), 4,4′-sec-butylidene diphenol, 4,4′-cyclohexylidene bisphenol, 4,4′-cyclohexylidene bis (2-ethyl phenol), 4-tert-butyl phenol, 4-phenyl phenol, 4-hydroxy diphenoxide, α-naphthol, β-naphthol, 3,5-xylenol, thymol, methyl-4-hydroxybenzoate, 4-hydroxyacetophenone, novolak phenol resins, 2,2′-thio bis(4,6-dichloro phenol), catechol, resorcin, hydroxynone, hydroquinone, pyrogallol, fluoroglycine, fluoroglycine carbonate, 4-tert-octyl catechol, 2,2′-methylene bis (4-chlorophenol), 2,2′-methylene bis (4-methyl-6-tert-butyl phenol), 2,2′-dihydroxy diphenyl, ethyl p-hydroxybenzoate, propyl p-hydroxybenzoate, butyl p-hydroxybenzoate, benzyl p-hydroxybenzoate, p-hydroxybenzoate-p-chlorobenzyl, p-hydroxybenzoate-o-chlorobenzyl, p-hydroxybenzoate-p-methylbenzyl, p-hydroxybenzoate-n-octyl, benzoic acid, zinc salicylate, 1-hydroxy-2-naphthoic acid, 2-hydroxy-6-naphthoic acid, 2-hydroxy-6-zinc naphthoate, 4-hydroxy diphenyl sulphone, 4-hydroxy-4′-chloro diphenyl sulfone, bis (4-hydroxy phenyl) sulfide, 2-hydroxy-p-toluic acid, 3,5-di-tert-zinc butyl salicylate, 3,5-di-tert-tin butyl salicylate, tartaric acid, oxalic acid, maleic acid, citric acid, succinic acid, stearic acid, 4-hydroxyphthalic acid, boric acid, thiourea derivative, 4-hydroxy thiophenol derivative, bis(4-hydroxyphenyl) acetate, bis(4-hydroxyphenyl)ethyl acetate, bis (4-hydroxyphenyl acetate-n-propyl, bis (4-hydroxyphenyl) acetate-n-butyl, bis (4-hydroxyphenyl) phenyl acetate, bis (4-hydroxyphenyl) benzyl acetate, bis (4-hydroxyphenyl) phenethyl acetate, bis (3-methyl-4-hydroxyphenyl) acetate, bis(3-methyl-4-hydroxyphenyl) methyl acetate, bis (3-methyl-4-hydroxyphenyl) acetate-n-propyl, 1,7-bis(4-hydroxyphenylthio) 3,5-dioxaheptane, 1,5-bis(4-hydroxyphenylthio) 3-oxaheptane, 4-hydroxy phthalate dimethyl, 4-hydroxy-4′-methoxy phenyl sulfone, 4-hydroxy-4′-ethoxy diphenyl sulfone, 4-hydroxy-4′-isopropoxy diphenyl sulfone, 4-hydroxy-4′-propoxy diphenyl sulfone, 4-hydroxy-4′-butoxy diphenyl sulfone, 4-hydroxy-4′-isopropoxy diphenyl sulfone, 4-hydroxy-4′-sec-butoxy diphenyl sulfone, 4-hydroxy-4′-tert-butoxy diphenyl sulfone, 4-hydroxy-4′-benzyloxy diphenyl sulfone, 4-hydroxy-4′-phenoxy diphenyl sulfone, 4-hydroxy-4′-(m-methyl benzoxy) diphenyl sulfone, 4-hydroxy-4′-(p-methyl benzoxy) diphenyl sulfone, 4-hydroxy-4′-(o-methyl benzoxy) diphenyl sulfone, 4-hydroxy-4′-(p-chloro benzoxy) diphenyl sulfone, 4-hydroxy-4′-oxyaryl diphenyl sulfone and the like.

4. The thermochromic dental material of claim 1, wherein the temperature sensitive thermochromic dye is bisphenol A.

5. The thermochromic dental material of claim 1, wherein the temperature sensitive thermochromic dye is from 0.01 to 10 wt %.

6. The thermochromic dental material of claim 1, wherein the polymeric dental composition is a dental composite, a dental acrylic, a polymer, or a plastic.

7. The thermochromic dental material of claim 1, wherein the polymeric dental composition comprises: between 20 to 90 wt % of one or more polymerizable monomers; 0.01 to 20 wt % of a polymerization initiator; and 5 to 90 wt % of a filler.

8. The thermochromic dental material of claim 1, wherein the polymeric dental composition is formed into a coating, a film, a filling, a reduction coping, a reduction coping, abutment, or a prosthetic.

9. A thermochromic coating comprising:
a polymeric dental composition; and
a temperature sensitive thermochromic dye in contact with the polymeric dental composition, wherein the temperature sensitive thermochromic dye is colorless at body temperature and undergoes a reversible color change at a temperature of between 99-160° F.

10. A method of making a temperature indicating dental material comprising the steps of:
providing one or more polymerizable monomers for use in a dental composition;
mixing one or more temperature sensitive thermochromic dye with the one or more polymerizable monomers;
forming a dental composition; and
curing the dental composition, wherein the temperature sensitive thermochromic dye is colorless at body temperature and undergoes a reversible color change at a temperature of between 99-160° F.

11. The method of claim 10 wherein the dental composition is a reduction coping, a reduction coping, abutment, or a prosthetic.

12. The method of claim 10 wherein the temperature sensitive thermochromic dye is a visual indicator a temperature of about 99, 100, 101, 102, 103, 105, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160° F., or above.

13. The method of claim 10 wherein the temperature sensitive thermochromic dye is selected from bisphenol A, tetrabromobisphenol A, gallnut acid, salicylic acid, 3-isopropyl salicylate, 3-cyclohexyl salicylate, 3-5-di-tert-butyl salicylate, 3,5-di-α-methyl benzyl salicylate, 4,4′-isopropylidenediphenol, 1,1′-isopropylidene bis (2-chlorophenol), 4,4′-isopropylene bis (2,6-dibromophenol), 4,4′-isopropylidene bis (2,6-dichlorophenol), 4,4′-isopropylidene bis(2-ethyl phenol), 4,4′-isopropylidene bis(2,6-dimethyl phenol), 4,4′-isopropylidene bis (2-tert-butyl phenol), 4,4′-sec-butylidene diphenol, 4,4′-cyclohexylidene bisphenol, 4,4′-cyclohexylidene bis (2-ethyl phenol), 4-tert-butyl phenol, 4-phenyl phenol, 4-hydroxy diphenoxide, a-naphthol, (3-naphthol, 3,5-xylenol, thymol, methyl-4-hydroxybenzoate, 4-hydroxyacetophenone, novolak phenol resins, 2,2′-thio bis(4,6-dichloro phenol), catechol, resorcin, hydroxynone, hydroquinone, pyrogallol, fluoroglycine, fluoroglycine carbonate, 4-tert-octyl catechol, 2,2′-methylene bis (4-chlorophenol), 2,2′-methylene bis (4-methyl-6-tert-butyl phenol), 2,2′-dihydroxy diphenyl, ethyl p-hydroxybenzoate, propyl p-hydroxybenzoate, butyl p-hydroxybenzoate, benzyl p-hydroxybenzoate, p-hydroxybenzoate-p-chlorobenzyl, p-hydroxybenzoate-o-chlorobenzyl, p-hydroxybenzoate-p-methylbenzyl, p-hydroxybenzoate-n-octyl, benzoic acid, zinc salicylate, 1-hydroxy-2-naphthoic acid, 2-hydroxy-6-naphthoic acid, 2-hydroxy-6-zinc naphthoate, 4-hydroxy diphenyl sulphone, 4-hydroxy-4′-chloro diphenyl sulfone, bis (4-hydroxy phenyl) sulfide, 2-hydroxy-p-toluic acid, 3,5-di-tert-zinc butyl salicylate, 3,5-di-tert-tin butyl salicylate, tartaric acid, oxalic acid, maleic acid, citric acid, succinic acid, stearic acid, 4-hydroxyphthalic acid, boric acid, thiourea derivative, 4-hydroxy thiophenol derivative, bis(4-hydroxyphenyl) acetate, bis(4-hydroxyphenyl)ethyl acetate, bis (4-hydroxyphenyl acetate-n-propyl, bis (4-hydroxyphenyl) acetate-n-butyl, bis (4-hydroxyphenyl) phenyl acetate, bis (4-hydroxyphenyl) benzyl acetate, bis (4-hydroxyphenyl) phenethyl acetate, bis (3-methyl-4-hydroxyphenyl) acetate, bis(3-methyl-4-hydroxyphenyl) methyl acetate, bis (3-methyl-4-hydroxyphenyl) acetate-n-propyl, 1,7-bis(4-hydroxyphenylthio) 3,5-dioxaheptane, 1,5-bis(4-hydroxyphenylthio) 3-oxaheptane, 4-hydroxy phthalate dimethyl, 4-hydroxy-4′-methoxy phenyl sulfone, 4-hydroxy-4′-ethoxy diphenyl sulfone, 4-hydroxy-4′-isopropoxy diphenyl sulfone, 4-hydroxy-4′-propoxy diphenyl sulfone, 4-hydroxy-4′-butoxy diphenyl sulfone, 4-hydroxy-4′-isopropoxy diphenyl sulfone, 4-hydroxy-4′-sec-butoxy diphenyl sulfone, 4-hydroxy-4′-tert-butoxy diphenyl sulfone, 4-hydroxy-4′-benzyloxy diphenyl sulfone, 4-hydroxy-4′-phenoxy diphenyl sulfone, 4-hydroxy-4′-(m-methyl benzoxy) diphenyl sulfone, 4-hydroxy-4′-(p-methyl benzoxy) diphenyl sulfone, 4-hydroxy-4′-(o-methyl benzoxy) diphenyl sulfone, 4-hydroxy-4′-(p-chloro benzoxy) diphenyl sulfone, 4-hydroxy-4′-oxyaryl diphenyl sulfone and the like.

14. The method of claim 10 wherein the temperature sensitive thermochromic dye is bisphenol A.

15. The method of claim 10 wherein the temperature sensitive thermochromic dye is from 0.01 to 10 wt %.

16. The method of claim 10, wherein the polymeric dental composition comprises: between 20 to 90 wt % of one or more polymerizable monomers; 0.01 to 20 wt % of a polymerization initiator; and 5 to 90 wt % of a filler.

17. A method of preventing cell and/or tissue damage and loss of the implant comprising the steps of:
(a) providing a thermochromic dental composition comprising: a polymeric dental composition; and a temperature sensitive thermochromic dye in contact with the polymeric dental composition, wherein the temperature sensitive thermochromic dye is colorless at body temperature and undergoes a reversible color change at a temperature of between 99-160° F.;
(b) placing the thermochromic dental composition in a patient;
(c) modifying the thermochromic dental composition, wherein heat is generated in the thermochromic dental composition and the temperature sensitive thermochromic dye changes color at a temperature;
(d) allowing the thermochromic dental composition to cool to below the temperature; and
(e) repeating (c) and (d).
